(19) United States
(12) Patent Application Publication
(10) Pub. No.: US 2006/0122081 A1
(43) Pub. Date: Jun. 8, 2006

(54) Cleaning composition

(75) Inventor: Niraj Dhansukhlal Mistry, Mumbai (IN)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: HINDUSTAN LEVER LIMITED, Mumbai (IN)

(21) Appl. No.: 10/559,588

(22) PCT Filed: May 17, 2004
(86) PCT No.: PCT/EP04/05274

§ 371 Date: Dec. 2, 2005

(30) Foreign Application Priority Data
Jun. 4, 2003 (IN) 573/MUM/03

(51) Int. Cl. A61K 8/00

(52) U.S. Cl. 510/130; 510/156

(57) ABSTRACT

A transparent liquid soap composition comprising: 5 to 25% by weight of soap, 2 to 20% by weight humectants, balance water, wherein the soap comprises 0.05 to 4% by weight of the composition castor oil soap or salt of ricinoleic acid, or the derivatives thereof.

The present invention relates to cleansing compositions in liquid/gel form. The present invention more particularly relates to liquid soap compositions prepared at low cost due to the inexpensive and widely available raw materials used, at the same time providing excellent cleaning properties, as well as high consumer appeal due the high transparency of the soap composition. It is possible by way of this invention to obtain/provide for excellent transparency to conventional compositions that are otherwise translucent/opaque.

Transparent soaps have aesthetic appeal, and are perceived to be milder than opaque soap compositions. Transparent soaps that are available in non-solid form e.g. in liquid or gel form also have high consumer appeal and are often used for specialised applications like hand wash and face wash. Liquid soaps are often used for out of home applications like during travel, at hotels and restaurants and where people are very conscious of hygiene, and where there is possibility of contamination at the wash place due to the large number of people using the soap.

Liquid soaps are often made by using alkali metal salts of fatty acids originating from vegetable or animal origin. Among the alkali metals, potassium is preferred over sodium as potassium fatty acids are more soluble in water thereby maintaining the liquid state in the formulation. However potassium salts are more expensive and there is a constant need to reduce cost by using more inexpensive salt like sodium salt of fatty acids. Of late, there is also a trend to incorporate non-soap detergents (NSD) and humectants such as glycerol that provide improved solubility in water but these materials tend to be more expensive.

There is a need felt in the art to incorporate high level of total fatty matter to ensure better cleaning, at the same time maintaining the fluid state and improving the transparency of the composition. It has been found by way of the present invention that small amount of soap made from castor oil based fatty acids, their precursors or derivatives in the total soap stock ensures high transparency in the composition, and allows for a wider formulation window like higher use of sodium soaps, lower use of non-soap detergents and humectants, and use of higher molecular weight fatty acid soaps.

U.S. Pat. No. 5,310,495 (Lever Brothers Company) relates to transparent bars said to be of exceptional clarity. The bar comprises a mixture of alkanolammonium and alkali metal fatty acid salts and a liquid solvent system including water and free alkanolamine. U.S. Pat. No. 2,820,768 (Fromont) and U.S. Pat. No. 4,206,069 (Borrello) also disclose the use of alkanolammonium soaps including free alkanolamine to provide for transparent soaps.

U.S. Pat. No. 4,310,432 (Lever Brothers Company, 1982) reports an aqueous liquid soap solution containing from 20% to 45% by weight of a sodium soap consisting essentially of a mixture of (A) at least one sodium soap of C₈ to C₁₄ saturated fatty acid and (B) at least one sodium soap of fatty acids selected from the group consisting essentially of C₁₆ to C₂₂ mono- and di-unsaturated fatty acids and mixtures thereof, the ratio of A to B being in from 4:1 to 1:4. Examples of component B as given in U.S. Pat. No. 4,310,432 are pure or technical grades of commercially available oleic acid, ricinoleic acid, fatty acids derived from castor oil and from rapeseed oil, and mixtures thereof.

The above patent describes a liquid soap composition comprising essentially of soluble soaps like sodium soaps of lower molecular weight saturated fatty acids (C₈ to C₁₄) as component A, and comprising high amounts of soap prepared from unsaturated fatty acids (C₁₆ to C₂₂) which include oleic acid, ricinoleic acid and fatty acids derived from castor or rapeseed oil.

The present inventors have now found that surprisingly low amounts of castor oil soap/salt of ricinoleic acid or their derivatives when incorporated in liquid/gel soap compositions provide for high transparency and excellent cleaning performance, and this property is not found when any of the other unsaturated fatty acids mentioned in U.S. Pat. No. 4,310,432 is used in such liquid/gel soap compositions even at high concentrations. The inventors have also found that this invention is applicable when the soap composition is prepared using total fatty matter of any molecular weight.

It is thus an object of the present invention to provide for a liquid/gel soap composition with excellent transparency and therefore high consumer appeal.

It is a further object of the present invention to provide for a transparent liquid/gel soap composition which comprises higher amount of soaps prepared from higher molecular weight fatty acids and sodium soaps which are more readily available, and are of lower cost and therefore are more economical to prepare.

It is yet another object of the present invention to provide for a transparent liquid/gel soap composition which can be prepared using lesser amounts of non-soap detergents or humectants like polyhydric alcohols which have been conventionally added to improve the solubility and transparency of liquid/gel soap compositions, thereby being able to prepare these compositions at lower cost.

It is a further object of the present invention to provide for a transparent liquid/gel soap which is prepared using very low amounts of unsaturated fatty acids, thereby giving enhanced stability of the soap composition.

The present invention thus provides for a transparent soap composition in liquid/gel form comprising, by weight of the composition:

5 to 25% of soap,

5-2 to 20% humectants,

optionally other conventional ingredients including non-soap detergents,

the balance being water

wherein the soap comprises 0.05 to 4% castor oil soap or salt of ricinoleic acid, or the derivatives thereof by weight of the composition.

According to a preferred aspect of the invention, there is provided a transparent liquid soap comprising, by weight of the composition

10% to 25% of alkali metal salts of C₈ to C₂₂ fatty acids,

2% to 20% of humectants chosen from polyhydric alcohols including gylcerol, sorbitol, polyethylene glycols, propylene glycols,

optionally other conventional ingradients including non-soap detergents,

the balance being water

wherein the total alkali metal salt of C₈ to C₂₂ fatty acids comprises 0.1% to 3% castor oil soap or alkali metal salt of ricinoleic acid or the derivatives thereof by weight of the composition.

According to another aspect of the invention, there is provided a process for preparing the transparent soap composition of the invention comprising the steps of:
a. mixing
(i) the total amount of salts of fatty acids including those prepared from castor oil or ricinoleic acid or their derivatives at the desired amounts
(ii) water
(iii) the humectants at the desired amounts
b. optionally adding to the above mixture suitable non-soap detergent actives
   to prepare the transparent soap composition as per the invention.

Throughout the specification, all parts are by weight unless otherwise specified.

By the word transparent is meant that the soap composition is capable of transmitting light there through.

The present invention relates to transparent liquid/gel soap compositions comprising 5 to 25% soap, humectants and water wherein 0.05% to 4% by weight of the composition is a castor oil soap/salt of ricinoleic acid or their derivatives.

The soap of the present invention are salts of fatty acids. Suitable fatty acids are the C₈-C₂₂ fatty acids. Fatty acids particularly suitable for the invention include lauric acid, myristic acid, palmitic acid and stearic acid. These can also be obtained from plant and/or animal sources, for example tallow fatty acids, palm fatty acids etc.

Resin acids, such as those present in tall oil are also suitable for the invention. Naphthenic acids may also be used for the invention.

The term soap refers to the salts of these fatty acids. Suitable cations include sodium, potassium, zinc, magnesium, alkyl ammonium and aluminium. Potassium and sodium are especially preferred cations. The invention is especially suitable for incorporation of sodium soaps along with potassium soaps. Sodium soaps at up to 50% of the total soap is possible.

It is also possible to saponify oils or their mixtures with fatty acids. Suitable oils for the invention include tallow, tallow stearines, palm oil, palm stearines, soya bean oil, fish oil, rice bran oil, sunflower oil, coconut oil, babassu oil and palm kernel oil. Especially preferred is hardened palm kernel oil.

The fatty acid soaps can also be prepared by a synthetic process e.g. by the oxidation of petroleum or by the hydrogenation of carbon monoxide by the Fischer-Tropsch process.

The soap content of the transparent soap composition is from 5% to 25%, more preferably from 10% to 25% and most preferably from 14% to 22%.

Soap prepared form castor oil or ricinoleic acids, or their derivatives is an essential component of the total fatty matter and is present in an amount of from 0.05% to 4% by weight of the total soap composition, preferably from 0.1% to 3% by weight of the composition.

Humectants are essential as per the soap composition of the invention, and include polyhydric alcohols. Polyhydric alcohols suitable for the invention include poly (ethylene glycol), propylene glycol, glycerol and sorbitol. Especially preferred is glycerol.

The polyhydric alcohol is preferably added in an amount of from 2% to 20%, more preferably from 5% to 15% by weight of the total soap composition.

As optional ingredients, non-soap detergent actives are preferably added during the process of the invention. They may be suitably added after the soap is mixed with water and the humectants. Non-soap detergent actives may be chosen from anionic, cationic, zwitterionic, amphoteric surfactants or their mixtures thereof.

The non-soap detergent active is generally chosen from an anionic, nonionic, cationic, zwitterionic detergent active or mixtures thereof. Preferably the amount of the non-soap detergent active does not exceed 20%.

Especially suitable anionic detergent active compounds are water soluble salts of organic sulphuric reaction products having in the molecular structure an alkyl radical containing from 8 to 22 carbon atoms, and a radical chosen from sulphonic acid or sulphur acid ester radicals and mixtures thereof.

Suitable nonionic detergent active compounds can be broadly described as compounds produced by the condensation of alkylene oxide groups, which are hydrophilic in nature, with an organic hydrophobic compound which may be aliphatic or alkyl aromatic in nature. The length of the hydrophilic or polyoxyalkylene radical which is condensed with any particular hydrophobic group can be readily adjusted to yield a water-soluble compound having the desired degree of balance between hydrophilic and hydrophobic elements.

Suitable amphoteric detergent-active compounds that optionally can be employed are derivatives of aliphatic secondary and tertiary amines containing an alkyl group of 8 to 18 carbon atoms and an aliphatic radical substituted by an anionic water-solubilizing group, for instance sodium 3-dodecylamino-propionate, sodium 3-dodecylaminopropane sulphonate and sodium N-2-hydroxydodecyl-N-methyltaurate. Suitable cationic detergent-active compounds are quaternary ammonium salts having an aliphatic radical of from 8 to 18 carbon atoms, for instance cetyltrimethyl ammonium bromide.

Suitable zwitterionic detergent-active compounds that optionally can be employed are derivatives of aliphatic quaternary ammonium, sulphonium and phosphonium compounds having an aliphatic radical of from 8 to 18 carbon atoms and an aliphatic radical substituted by an anionic water-solubilising group, for instance 3-(N-N-dimethyl-N-hexadecylammonium), propane-1-sulphonate betaine, 3-(dodecylmethyl sulphonium) propane-1-sulphonate betaine and 3-(cetylmethylphosphonium) ethane sulphonate betaine.

Further examples of suitable detergent-active compounds are compounds commonly used as surface-active agents given in the well-known textbooks “Surface Active Agents”, Volume I by Schwartz and Perry and “Surface Active Agents and Detergents”, Volume-II by Schwartz, Perry and Berch.

Salts are preferably added to the soap composition of the invention. Suitable salts include sodium and potassium salts. Sodium chloride is an especially preferred salt and is preferably used in an amount of from 0.1% to 2%.

Other optional ingredients like anti-oxidants, perfumes, polymers, thickening agents, chelating agents, colourants, deodorants, dyes, emollients, moisturisers, enzymes, foam boosters, germicides, anti-microbials, lathering agents, pearlescers, skin conditioners, solvents, stabilisers, superfatting agents, sunscreens etc. may be added in suitable amounts in the process of the invention, provided the transparency of the soap is retained. Preferably, the ingredients are added after the essential ingredients are mixed in the composition.

A soap composition as per the invention is prepared starting with the soap having the desired ratio of castor fatty matter: other fatty matter, followed by addition of desired amounts of water, humectants and other optional ingredients.

Alternately, castor soap may be mixed with the other soaps in the desired ratio along with water, humectants and other optional ingredients to prepare the highly transparent liquid/gel soap composition of the invention.

### EXAMPLES

The invention will be further described by the following illustrative non-limiting examples. All parts therein are by weight unless otherwise specified.

The transmittance of the compositions was measured by the following method.

The soap composition was taken as such and the transmittance of the product was measured in a 1 ml cell at 600 nm at 25° C. using a path length of 1 cm in a Shimadzu UV-160A spectrophotometer, using water as the reference.

Liquid soap compositions comprising potassium stearate and potassium laurate were prepared, and are summarized in Table-1. Small amounts of other fatty acid soaps (castor or oleate) were added to the compositions. The soap compositions were made up to 100% with water. The transparency of the solutions as measured using the transmittance of light is also summarized in table-1.

TABLE 1

| | Potassium | Potassium | Potassium | Castor oil | | |
|---|---|---|---|---|---|---|
| Example | stearate, | Laurate, | oleate | potassium | Glycerin, | |
| No. | wt % | wt % | wt % | soap, wt % | wt % | % transmittance |
| A | 10 | 10 | — | — | 10 | 0.3 |
| B | 10 | 10 | 1 | — | 10 | 0.2 |
| 1 | 10 | 10 | — | 1 | 10 | 67.2 |

Similar compositions were prepared using potassium laurate and potassium palmitate soaps. The compositions were made up to 100% with water and the data is presented in Table-2

TABLE 2

| | Potassium | Potassium | | Castor oil | | |
|---|---|---|---|---|---|---|
| Example | palmitate, | Laurate, | Potassium | potassium | Glycerin, | |
| No. | wt % | wt % | oleate, wt % | soap, wt % | wt % | % transmittance |
| C | 10 | 10 | — | — | 5 | 0.8 |
| D | 9.5 | 9.5 | 1 | — | 5 | 0.7 |
| E | 7.5 | 7.5 | 5 | — | 5 | 0.8 |
| F | 5 | 5 | 10 | — | 5 | 4.2 |
| 2 | 10 | 10 | — | 2 | 5 | 86.6 |
| G | 10 | 10 | — | — | 10 | 11.5 |
| 3 | 10 | 10 | — | 1 | 10 | 85.7 |

Experiments were also conducted using mixed sodium/potassium (50:50) soap systems with the compositions made up to 100% with water and the data is presented in table-3.

TABLE 3

| | Na/K | Na/K | Castor oil | | |
|---|---|---|---|---|---|
| Example | Stearate, | Laurate, | potassium | Glycerin, | % |
| No. | wt % | wt % | soap, wt % | wt % | transmittance |
| H | 10 | 10 | — | 10 | 0.3 |
| 4 | 10 | 10 | 1 | 10 | 90.2 |

The data in table-1 to 3 indicate that when castor soap in small amounts is added to soap compositions having poor transparency (Examples—A, C, G), the transparency of such compositions increases dramatically (Examples—1, 2, 3). Such behaviour is not seen when oleate soap even at very high concentration is added to the compositions (Examples—B, D, E, F). It is also possible by way of the present invention to include much higher amounts of more insoluble soaps (e.g sodium stearate/laurate soaps) to the total soap stock to provide for cleaning compositions with excellent transparency (example 4).

Further experiments were done to check effect of using known humectants like propylene glycols/poly ethylene glycol (PEG) at high levels in soap compositions comprising more amounts of soluble soaps e.g myristate and laurate. The following samples, summarized in table-4, were prepared with the composition made up to 100% with water. The visual appearance of the samples was noted at 25° C. and at 4° C. and the data is summarized in table-4.

TABLE 4

| Example No. | I | J | 5 | K | 6 |
|---|---|---|---|---|---|
| Potassium palmitate, wt % | 5 | 5 | 5 | 4.0 | 4.0 |
| Potassium Laurate, wt % | 12 | 12 | 12 | 9.6 | 9.6 |
| Potassium myristate, wt % | 8 | 8 | 8 | 6.4 | 6.4 |
| Castor soap, wt % | — | — | 1 | | 1 |
| Propylene Glycol, wt % | 5 | 15 | 15 | 15 | 10 |
| PEG-1500, wt % | — | — | — | — | 5 |
| Sample appearance, visual, | Clear | Clear | Clear | Clear | Clear |
| 25° C. | | | | | |
| Sample appearance, visual, | Hazy | Milky | Clear | Milky | Clear |
| 4° C. | | | glass | | glass |
| | | | like | | like |

The data in Table 4 indicates that samples having high levels of humectants up to 15% (Examples I, J, K) which give good clarity at around room temperatures give poor clarity at cold conditions. When small amount of castor soap was added to similar samples, they give a very good visual appearance of clarity over a wide range of temperatures. (Examples 5 and 6)

Further experiment were conducted using other known solubilization enhancers like sodium citrate and polyacrylic acid with and without castor soap and the visual appearance was noted at 25° C. and 4° C. The data is summarized in Table 5.

TABLE 5

| Example No. | L | M | N | 7 | 8 | 9 | 10 |
|---|---|---|---|---|---|---|---|
| Potassium palmitate, wt % | 5 | 5 | 5 | 5 | 5 | 5 | 5 |
| Potassium Laurate, wt % | 12 | 12 | 12 | 12 | 12 | 12 | 12 |
| Potassium myristate, wt % | 8 | 8 | 8 | 8 | 8 | 8 | 8 |
| Castor soap, wt % | — | — | — | 0.5 | 1.0 | 1.5 | 2.0 |
| Sodium citrate, wt % | 0.5 | — | — | — | — | — | — |
| Poly acrylic acid, wt % | — | 1.0 | — | — | — | — | — |
| Propylene glycol, wt % | 15 | 15 | 5 | 5 | 5 | 5 | 5 |
| Glycerine, wt % | — | — | 10 | 10 | 10 | 10 | 10 |
| Sample appearance, visual, 25° C. | Clear | Clear | Clear | Clear | Clear | Clear | Clear |
| Sample appearance, visual, 4° C. | Milky | Milky | Hazy | Clear | Clear | Clear | Clear |
| | | crystals | opaque | glass | glass | glass | glass |
| | | | | like | like | like | like |

The data in Table 5 indicates that samples L and M which show good clarity at around room temperatures show poor clarity at cold conditions when known solubilizers were used. Samples 7 to 10 containing castor soap over a wide range of concentrations indicate good clarity over a wide temperature range compared to the sample without castor (Example—N).

1. A transparent liquid soap composition comprising:
to 25% by weight of soap,
2 to 20% by weight humectants,
balance water;
wherein the soap comprises 0.05 to 4% by weight of the composition castor oil soap or salt of ricinoleic acid, or the derivatives thereof.

2. The transparent liquid soap of claim 1 wherein the soap comprises alkali metal salts of C₈ to C₂₂ fatty acids.

3. The transparent liquid soap of claim 1 wherein the soap is present in 14 to 22% by weight of the composition.

4. The transparent liquid soap of claim 1 wherein the soap comprises 0.1 to 3% by weight of the composition castor oil soap or salt of ricinoleic acid or the derivatives thereof.

5. The transparent liquid soap of claim 1 wherein the humectant is selected from polyhydric alcohols including glycerol, sorbitol, polyethylene glycols, propylene glycols and mixtures thereof.

6. The transparent liquid soap of claim 5 wherein the polyhdyric alcohol is glycerol.

7. The transparent liquid soap of claim 1 wherein the humectant is present in 5 to 15% by weight of the composition.

8. The transparent liquid soap of claim 1 further comprising not more than 20% by weight of a non-soap detergent active.

9. A process for preparing the transparent soap composition of claim 1 comprising mixing
(i) the total amount of salts of fatty acids including those prepared from castor oil or ricinoleic acid or their derivatives;
(ii) water; and
(iii) the humectants.

10. The process of claim 9 further comprising the step of adding to the above mixture suitable non-soap detergent actives.
